(19) United States
(12) Patent Application Publication
(10) Pub. No.: US 2012/0118294 A1
(43) Pub. Date: May 17, 2012

(54) TRACHEAL TUBE WITH REINFORCED PROXIMAL EXTENSION

(75) Inventors: Paul Waldron, Galway (IE); Declan Kiernan, Co. Longford (IE); Seamus Maguire, Athlone (IE)

(73) Assignee: Nellcor Puritan Bennett LLC, Boulder, CO (US)

(21) Appl. No.: 12/944,172

(22) Filed: Nov. 11, 2010

(51) Int. Cl. A61M 16/04

(52) U.S. Cl. 128/207.14

(57) ABSTRACT

A tracheal tube includes a proximal extension between a flange member and a connector. The extension may allow for easier access to the connector. The extension is reinforced to limit bending and kinking. Reinforcement may be accomplished by a helical element that is embedded in the extension, such as between an inner cannula and an outer cover. The outer cover may be molded with a connector after placement of the reinforcing member.

### BACKGROUND

The present disclosure relates generally to the field of tracheal tubes and, more particularly, to a tracheal tube having a reinforced proximal extension.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired, For short-term ventilation or during certain surgical procedures, endotracheal tubes may be inserted through the mouth to provide oxygen and other gasses to a patient. For other applications, particularly when longer-term intubation is anticipated, tracheostomy tubes may be preferred. Trachostomy tubes are typically inserted through an incision made in the neck of the patient and through the trachea. A resulting stoma is formed between the tracheal rings below the vocal chords. The tracheostomy tube is then inserted through the opening. In general, two procedures are common for insertion of tracheostomy tubes, including a surgical procedure and a percutaneous technique.

Such tubes may include an inflatable balloon cuff, or may be cuffless. In both cases, a connector is typically provided at an upper or proximal end where the tube exits the patient airway. Standard connectors have been developed to allow the tube to then be coupled to artificial ventilation equipment to supply the desired air or gas mixture to the patient, and to evacuate gasses from the lungs.

One difficulty that arises in the use of tracheal tubes, and tracheostomy tubes in particular, is in the connection and manipulation of the proximal end of the tube. For example, endotracheal tubes may be fairly large in diameter, depending upon the size and age of the patient. Tracheostomy tubes, on the other hand, are typically kept fairly small to accommodate the routing of the tube through the passageway formed in the neck and trachea of the patient. This small size may lead to issues when a connection is made to ventilation equipment or when the tubes must be manipulated or held in place while a connection is fitted over a standard mating connector end formed on the tube. The orientation of the mating connector on the tube may make such connections difficult, and may cause movement of the tube in the patient during normal patient activity, even if somewhat constrained, and during movement of the patient, such as for changing of bed linens, surgical and imaging procedures, and so forth. In conventional tubes, the connector is provided relatively close to the neck of the patient, making connections somewhat more difficult.

There is a need, therefore, for improved tracheal tubes, and particularly for improved endotracheal tubes. It would be desirable to provide a tube that allows for greater facility in making and changing connections with ventilation equipment while reducing the potential for bending or kinking of the tube at a proximal end.

### BRIEF DESCRIPTION

Provided herein is a novel tracheal tube designed to respond to such needs. The tube allows for extension of a proximal end beyond a point where it exits the patient. In a tracheostomy tube embodiment, for example, a flange member fits adjacent to the neck of a patient and an extension is provided between this member and a standard connector. The extension is reinforced to allow for manipulation of the tube and the connector while reducing the potential for bending or kinking.

Thus, in accordance with a first aspect, a tracheal tube assembly comprises a cannula configured to be positioned in a patient airway, a flange member secured about the cannula, and a connector. A proximal extension is provided that comprises a reinforced tubular member extending between the flange member and the connector. The cannula, the proximal extension and the connector form a contiguous passageway for exchanging gas with the patient airway in operation.

In accordance with another aspect, a tracheal tube assembly comprises a cannula configured to be positioned in a patient airway, and a flange member secured about the cannula. A proximal extension comprises an inner tubular member contiguous with the cannula, a reinforcing member disposed about the inner tubular member, and an outer cover disposed about the inner tubular member and the reinforcing member. A connector is secured to the proximal extension. The cannula, the proximal extension and the connector form a contiguous passageway for exchanging gas with the patient airway in operation.

This disclosure also provides a method for making a tracheal tube that comprises inserting a cannula through a flange member, and disposing a reinforcing member about an upper portion of the cannula extending through an upper surface of the flange member. An outer cover is disposed about the cannula and the reinforcing member, and a connector is disposed at an upper end of the cannula.

### BRIEF DESCRIPTION OF THE DRAWINGS

Advantages of the disclosed techniques may become apparent upon reading the following detailed description and upon reference to the drawings in which:

FIG. 1 is a perspective view of an exemplary tracheal tube in accordance with aspects of the present techniques;

FIG. 2 is a similar prospective view of certain functional parts of the tube shown in FIG. 1 prior to final assembly;

FIG. 3 is an assembly view of the same tube illustrating a proximal extension and a reinforcing element within a later-formed connector; and

FIG. 4 is sectional view through the final product shown in FIG. 1 illustrating the various parts of the assembly, particularly those of the reinforced proximal extension.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

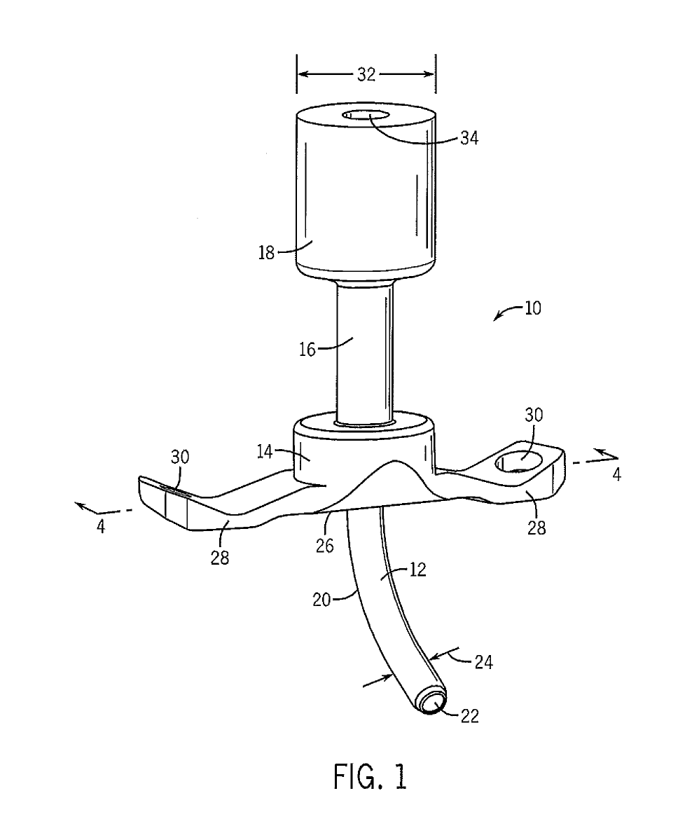

A tracheal tube in accordance with aspects of the present disclosure is illustrated in FIG. 1. The tracheal tube assembly 10 represented in the figures is a tracheostomy tube, although aspects of the present disclosure could be applied to other tracheal tube structures, such as endotracheal tubes. The application to a tracheostomy tube is particularly important, however, insomuch as such tubes tend to be smaller and more prone to bending, particularly if extensions are provided between the base of the tube and a connection as described below.

The assembly 10 includes a cannula 12 extending from a flange member 14. Above the flange member, a proximal extension 16 is formed that terminates in a connector 18. In use, the cannula 12 is placed through an opening formed in the neck and trachea of a patient, and extends into the patient airway. The embodiment illustrated the figures is free of outer seals or cuffs, although in practice the designs and techniques described in the present discussion may be used with a wide range of tube designs, including tubes having one or more sealing cuffs around the cannula 12. Moreover, the cannula may include a single tube or nested tubes, depending upon the assembly design. The cannula 12 in the illustrated embodiment forms an arcuate tube 20 through which a passageway 22 is provided. The cannula has an outer dimension 24 allowing it to fit easily through an incision made in the neck and trachea of the patient. In practice, a range of such tubes may be provided to accommodate the different contours and sizes of patients and patient airways. Such tube families may include tubes designed for neonatal and pediatric patients as well as for adults. By way of example only, outer dimension 24 of the tube 20 may range from 4 mm to 16 mm.

The cannula 12 enters the flange member 14 along a lower face 26. When in use, the face 26 will generally be positioned against the neck of a patient, with the cannula extending through an opening formed in the neck and trachea, A pair of side flanges 28 extend laterally and serve to allow a strap or retaining member (not shown) to hold the tube assembly in place on the patient. In the illustrated embodiment, apertures 30 are formed in each side flange to allow the passage of such a retaining device. In many applications the flange member may be taped in place as well.

The proximal extension 16 extends from the flange member 14 and allows for ease of access to the connector 18. The extension essentially forms a neck or tubular section between the upper surface of the flange member and the lower surface of the connector. In presently contemplated embodiments, the extension between these surfaces may have a length ranging from approximately 15 mm to approximately 60 mm, although other lengths may be envisaged. Similarly, in the illustrated embodiments the proximal extension is generally straight and cylindrical. In other configurations, however, the extension could be formed with bends, radiuses, and so forth.

The connector 18 is formed in accordance with industry standards to permit and facilitate connection to ventilating equipment (not shown). By way of example, standard outer dimensions may be provided as indicated at reference numeral 32 that allow a mating connector piece to be secured on the connector shown. By way of example, a presently contemplated standard dimension 32 accommodates a 15 mm connector, although other sizes and connector styles may be used. An aperture 24 is formed in the connector 18 and is contiguous with the proximal extension 16 and the passageway 22 formed in the cannula. In use, then, air or other gas may be supplied through the connector, the proximal extension and the cannula, and gasses may be extracted from the patient.

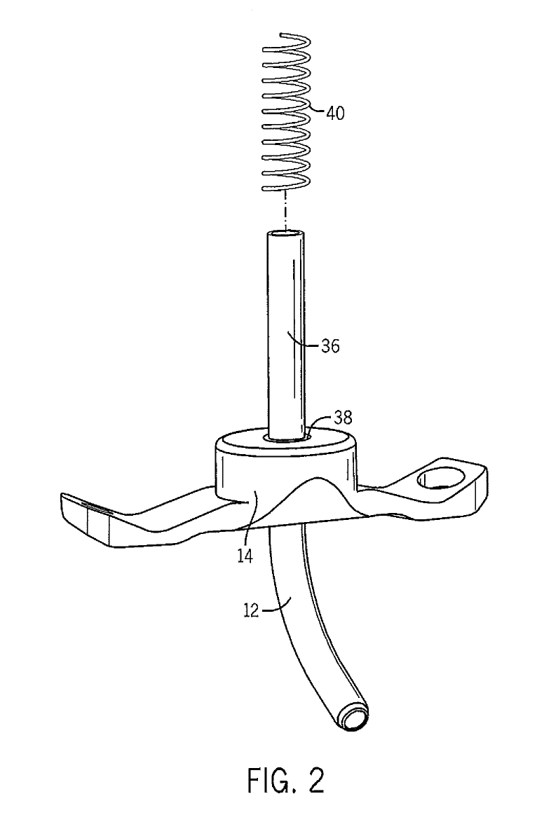

FIG. 2 illustrates elements of the tracheal tube assembly during assembly. In the illustrated embodiment the cannula 12 has an inner cannula section 36 that extends through a passageway 38 formed in the flange member 14. In presently contemplated embodiments the cannula is formed as a single piece such that the tube forming the cannula may be slipped through the passageway 38 to a point where the inner cannula 36 extends a desired height above the upper surface of the flange member. Where the cannula 12 is bent or arcuate as in the illustrated example, the cannula is angularly positioned with respect to the flange member such that the are of the cannula is in a desired orientation with respect to the side flanges of the flange member.

Due to the length of the inner cannula 36, a reinforcing member 40 is disposed around the inner cannula to provide reinforcement against bending, kinking, and deformation. In the illustrated embodiment, the reinforcing member 40 is a helical or coil-like piece that is pressed or slips around the outer surface of the inner cannula and is lowered to a position just above or in contact with the upper surface of the flange member. In a presently contemplated embodiment, the cannula 12, including the inner cannula 36, is made of polyvinylchloride, and the reinforcing member 40 is made of nylon. The materials used for these components may vary, however, and acceptable materials may include, by way of example, a PEBAX silicone, or polyurethane could be used for the cannula and phosphor bronze, stainless steel or nitinol could be used for the spring. In certain embodiments, it may be preferred that the reinforcing member is made of a non-ferromagnetic material such that the entire tube assembly may be left in place during certain imaging procedures, such as magnetic resonance imaging.

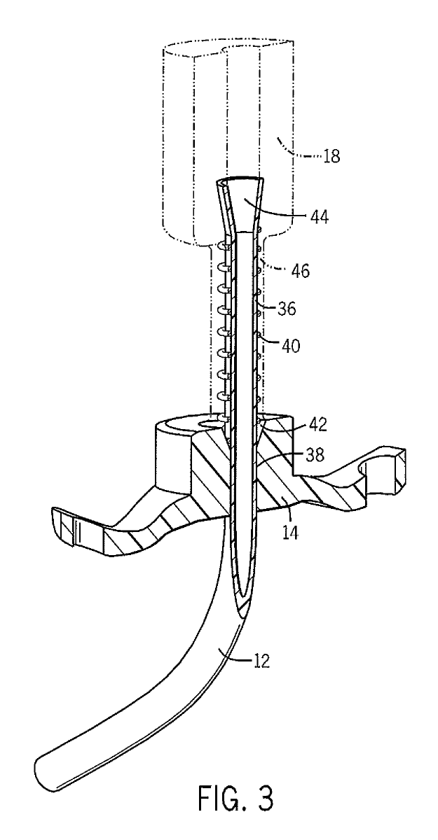

FIG. 3 illustrates an intermediate stage in the manufacture of the tracheal tube assembly in accordance with a presently contemplated embodiment. In this embodiment, a tapered opening 42 is formed in the flange member 14. While a flange member may be an assembly of components, in the illustrated embodiment it is a single molded piece, such that the tapered opening 42 can be formed by the die in which the flange member is molded. The passageway 38 is also formed in a similar manner through the flange member, and the cannula 12 is installed as best illustrated in FIG. 2 by pressing the upper end of the inner cannula portion through the passageway in the flange member.

After installation of the reinforcing member 40 about the inner cannula 36, the upper region 44 of the inner cannula may be flared or otherwise plastically deformed to facilitate transition in dimensions and to aid in retaining the structure integrally in the final assembled tracheal tube. In the illustrated embodiment, for example, a dimensional transition in the inner diameter of the cannula 12 may be effectuated by the flared upper end 44 of the inner cannula 36, such that at an upper end of the inner cannula 36 the internal diameter matches that of the opening 34 formed in the connector (see FIG. 1). The flaring of the inner cannula 36, where performed, may be accomplished by any range of tooling, such as a tapered flare tooling (not shown) that is pressed into the upper end of the inner cannula with the inner cannula and other components held in place in a fixture.

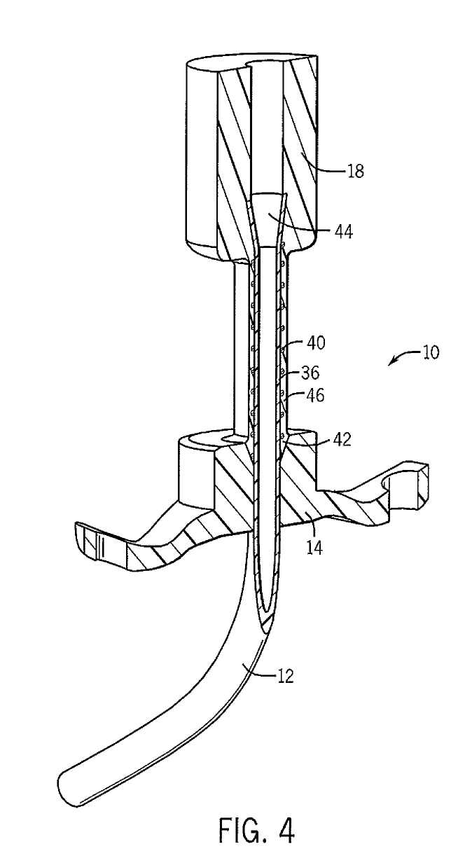

Thereafter, the connector 18 may be installed. The presently contemplated embodiment illustrated, the connector 18 is integrally formed with a molded outer cover 46. In this embodiment, the partially completed tube, including the flange member 14, the cannula 12 with the inner cannula 36, and the reinforcing member 40 are placed in a mold and a synthetic plastic material is injected to form the outer cover 46 and the connector 18 in a single operation. Other embodiments may call for inserts, collars, transition elements, and so forth in the connector 18 that may be inserted into a mold during the same process, or that may be inserted, assembled or affixed in separate operations. In a presently contemplated embodiment, the overmolded outer cover 46 and the connecter 18 are formed of polyvinylchloride or PEBAX, although other suitable materials may be employed. Once completed, the structure may appear as illustrated in FIG. 4, which is a sectional view of the finished tracheal tube assembly shown in FIG. 1. It should be noted that in this embodiment, the material forming the outer cover 46 fills the tapered opening 42 in the flange member, if provided, and also serves to provide structural integrity to the inner cannula 36 and reinforcing member 40 that form the proximal extension.

What is claimed is:

1. A tracheal tube assembly comprising:
a cannula configured to be positioned in a patient airway;
a flange member secured about the cannula;
a connector; and
a proximal extension comprising a reinforced tubular member extending between the flange member and the connector, wherein the cannula, the proximal extension and the connector form a contiguous passageway for exchanging gas with the patient airway in operation.

2. The assembly of claim 1, wherein the proximal extension comprises an inner tubular member and a reinforcing member disposed about the inner tubular member.

3. The assembly of claim 2, wherein the proximal extension comprises an outer cover disposed about the inner tubular member and the reinforcing member.

4. The assembly of claim 3, wherein the outer cover is overmolded around the inner tubular member and the reinforcing member.

5. The assembly of claim 4, wherein the outer cover is integrally molded with the connector.

6. The assembly of claim 2, wherein the reinforcing member comprises a helical element wrapped around the inner tubular member.

7. The assembly of claim 6, wherein the helical element is made of a synthetic plastic material.

8. The assembly of claim 7, wherein the synthetic plastic material comprises nylon.

9. The assembly of claim 8, wherein the inner tubular member is flared in a region where the inner tubular member joins the connector.

10. The assembly of claim 1, wherein the proximal extension has a length, between an upper surface of the flange member and a lower surface of the connector, of between approximately 15 mm and approximately 60 mm

11. A tracheal tube assembly comprising:
a cannula configured to be positioned in a patient airway;
a flange member secured about the cannula;
a proximal extension comprising an inner tubular member contiguous with the cannula, a reinforcing member disposed about the inner tubular member, and an outer cover disposed about the inner tubular member and the reinforcing member; and
a connector secured to the proximal extension, wherein the cannula, the proximal extension and the connector form a contiguous passageway for exchanging gas with the patient airway in operation.

12. The assembly of claim 11, wherein the outer cover is overmolded around the inner tubular member and the reinforcing member.

13. The assembly of claim 12, wherein the outer cover is integrally molded with the connector.

14. The assembly of claim 11, wherein the reinforcing member comprises a helical synthetic plastic element wrapped around the inner tubular member.

15. The assembly of claim 11, wherein the inner tubular member is flared in a region where the inner tubular member joins the connector.

16. The assembly of claim 11, wherein the proximal extension has a length, between an upper surface of the flange member and a lower surface of the connector, of between approximately 15 mm and approximately 60 mm.

17. A method for making a tracheal tube, comprising:
inserting a cannula through a flange member;
disposing a reinforcing member about an upper portion of the cannula extending through an upper surface of the flange member;
disposing an outer cover about the cannula and the reinforcing member; and
disposing a connector at an upper end of the cannula.

18. The method of claim 17, wherein the reinforcing member comprises a generally helical member that wraps around the upper portion of the cannula.

19. The method of claim 17, wherein the outer cover and the connector are overmolded over the upper portion of the cannula and the reinforcing member.

20. The method of claim 17, comprising flaring an end of the upper portion of the cannula in a region in which the connector is disposed.
